(19) United States
(12) Patent Application Publication
(10) Pub. No.: US 2009/0230167 A1
(43) Pub. Date: Sep. 17, 2009

(54) Endostapler Biasing Mechanism

(75) Inventors: Jia-Hua Xiao, Santa Rosa, CA (US); Eric Meyer, Andover, MN (US); Jeffrey Sandstrom, Forest Lake, MN (US); Damian Jelich, Cottage Grove, MN (US); Trevor Greenan, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/049,531

(22) Filed: Mar. 17, 2008

(51) Int. Cl. A61B 17/068

(52) U.S. Cl. 227/175.1; 128/898

(57) ABSTRACT

An endostapler delivery system includes a biasing mechanism to offset or counter forces generated by a stapling device and therefore prevent the stapling device from moving during the firing of the staple. The delivery system includes a catheter having at least one lumen extending there through for receiving the stapling device. The biasing mechanism is an expandable biasing cage having a dome or semi-circular expanded shape provided at the distal portion of the catheter. When expanded, the biasing cage does not block or occlude a vessel, thereby allowing blood flow to continue during the stapling procedure. The biasing cage may include a plurality of ribbons or strands that extend generally parallel to the blood flow when expanded in situ, a mesh or braided structure, or a plurality of ribbons or strands that extend generally parallel to the blood flow when expanded in situ and a mesh or braided structure placed over the plurality of ribbons.

### FIELD OF THE INVENTION

The present invention relates generally to endostapler delivery systems employed in the treatment of vascular disease. More particularly, the present invention relates to endostapler delivery systems including a biasing mechanism for use in the fixation of grafts to the walls of vessels.

### BACKGROUND

In modern medical practice, it is sometimes desirable to pass a stapling device into or through the wall of a luminal anatomical structure (e.g., a blood vessel or other anatomical conduit) for the purpose of attaching an article (e.g., an endoluminal, extraluminal or transluminal graft) or other apparatus to the wall of the anatomical structure.

Examples of medical procedures wherein it is desirable to anchor or attach a graft or other apparatus to the wall of a blood vessel or other luminal anatomical conduit include certain endovascular grafting procedures wherein a tubular graft is placed within the lumen of an aneurysmal blood vessel to create a neo-lumen or artificial flow conduit through an aneurysm, thereby reducing if not completely eliminating the exertion of blood pressure on the aneurysm and allowing the aneurysmal sac to subsequently become stagnant and transform to granulation tissue. These endovascular grafting procedures have heretofore been used to treat aneurysms of the abdominal aorta, as well as aneurysms of the descending thoracic aorta. Endovascular grafts used typically incorporate or are combined with one or more radially expandable stents which are radially expanded in situ to anchor the tubular graft to the wall of the blood vessel at sites upstream and downstream of the aneurysm. Thus, the grafts are typically held in place by mechanical engagement, tissue ingrowth, and friction via the self-expanding or balloon expandable stents. The grafts may also be affixed to vessels with hooks or barbs.

However, in the event that the force provided by these stent(s) fails to establish sound mechanical and/or frictional engagement with the blood vessel wall, the graft may undergo undesirable migration or slippage, or blood may leak into the aneurysmal sac (sometimes referred to as an “endoleak”). Thus, in view of the above-mentioned undesirable complications associated with the use of radially expandable stents to mechanically and/or frictionally anchor a graft or other apparatus to the wall of a blood vessel (or other luminal anatomical structure) there exists a need in the art for the development of new endoluminal attachment devices which may be used to attach the ends of a endoluminal tube graft (or other article) to the surrounding wall of a blood vessel or other tubular anatomical conduit, thereby ensuring sound and permanent placement of the graft or other article.

### BRIEF SUMMARY OF THE INVENTION

Embodiments described herein relate to an endostapler delivery system for delivering a stapling device through a body lumen. The system includes a catheter shaft including a proximal portion and a distal portion, the catheter shaft defining a first lumen having an exit port disposed at the distal portion of the catheter shaft and a second lumen having a side exit port disposed at the distal portion of the catheter shaft. The first and second lumens extend side-by-side from the proximal portion to the distal portion of the catheter shaft, and the first lumen of the catheter shaft is of a sufficient size such that the stapling device may be advanced there through. An expandable biasing cage is disposed within the second lumen of the catheter shaft. An actuator is disposed at the proximal portion of the catheter shaft, wherein the actuator is adapted to expand the biasing cage to a dome shape extending outside of the catheter shaft via the side exit port such that the biasing cage abuts a vessel wall of the body lumen and/or a graft implanted within the body lumen. The biasing cage when expanded does not block or occlude the body lumen such that blood may flow there through.

### BRIEF DESCRIPTION OF DRAWINGS

The foregoing and other features and advantages will be apparent from the following description of embodiments as illustrated in the accompanying drawings. The accompanying drawings, which are incorporated herein and form a part of the specification, further serve to explain the principles used in the embodiments. The drawings are not to scale.

FIG. 1 is a schematic isometric view of an endostapler delivery system.

FIG. 2 is a cross-sectional view of a vessel within which the endostapler delivery system in FIG. 1 (only the end of which can be seen) is configured to position the stapler opening of the system adjacent the vessel wall for attaching an endoluminal graft to a vessel wall.

FIG. 3 is a sectional side view of the endostapler delivery system of FIG. 1, wherein a ribbon of a biasing cage of the endostapler delivery system is in an unexpanded configuration.

FIG. 4 is a sectional side view of the endostapler delivery system of FIG. 1, wherein the ribbon of the biasing cage of the endostapler delivery system is in an expanded configuration.

FIG. 5A is a cross-sectional view of the endostapler delivery system of FIG. 1.

FIG. 5B is a cross-sectional view of another embodiment of the endostapler delivery system of FIG. 1.

FIG. 6 is a pictorial view of a distal portion of the endostapler delivery system illustrated in FIG. 1, wherein the biasing cage of the endostapler delivery system in an expanded configuration.

FIG. 7 is a schematic isometric view of another embodiment of an endostapler delivery system.

FIG. 8 is a cross-sectional view of a vessel within which the endostapler delivery system in FIG. 7 (only the end view of which can be seen) is configured to position the stapler opening of the system adjacent the vessel wall.

FIG. 9A is a sectional side view of the endostapler delivery system of FIG. 7, wherein a plurality of braided elements of a biasing cage of the endostapler delivery system are in an unexpanded configuration.

FIG. 9B is a sectional side view of the endostapler delivery system of FIG. 7, wherein the braided elements of the biasing cage of the endostapler delivery system are in an expanded configuration.

FIG. 10 is a side pictorial view of a distal portion of the endostapler delivery system of FIG. 7, wherein the braided elements of the biasing cage of the endostapler delivery system are in an expanded configuration.

FIG. 11 is a schematic isometric view of another embodiment of an endostapler delivery system.

FIG. 12 is a cross-sectional view of a vessel within which the endostapler delivery system of FIG. 11 (only the end view of which can be seen) is configured to position the stapler opening of the system adjacent the vessel wall.

FIG. 13A is a sectional side view of the endostapler delivery system of FIG. 11, wherein the braided elements and ribbon of a biasing cage of the endostapler delivery system are in an unexpanded configuration.

FIG. 13B is a sectional side view of the endostapler delivery system of FIG. 11, wherein the braided elements and ribbon of a biasing cage of the endostapler delivery system are in an expanded configuration.

FIG. 14 is a top pictorial view of a distal portion of the endostapler delivery system of FIG. 11, wherein the braided elements and ribbon of the biasing cage of the endostapler delivery system are in an expanded configuration.

FIG. 15 is a side view illustration of a distal portion of the endostapler delivery system of FIG. 11, wherein the braided elements and ribbon of the biasing cage of the endostapler delivery system are in an expanded configuration.

FIG. 16 is a sectional side view of an endostapler delivery system according to another embodiment, wherein a biasing cage of the endostapler delivery system is in an unexpanded configuration.

### DETAILED DESCRIPTION

Specific embodiments are now described with reference to the figures, wherein like reference numbers indicate identical or functionally similar elements. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician.

The following detailed description is merely exemplary in nature and is not intended to limit the number of possible variations of embodiments according to the invention. Although the description of embodiments is in the context of treatment of blood vessels such as the coronary, carotid and renal arteries, the embodiments may also be used in any other body passageways where it is deemed useful.

Embodiments described relate to an endostapler delivery system having a biasing mechanism to offset or counter forces generated by a stapling device. A delivery system includes a catheter having at least one lumen extending there through for receiving the stapling device. A biasing mechanism is an expandable biasing cage having a dome or semi-circular expanded shape provided at the distal portion of the catheter. When expanded, the biasing cage forces the stapling device against a receiving area of a vessel wall and/or graft where a staple is to be fired. Further, the biasing cage prevents the stapling device from moving during the firing of the staple. An expanded biasing cage does not block or occlude the vessel in order to allow blood flow to continue during the stapling procedure. Thus, the biasing cage allows blood perfusion at all times while simultaneously offsetting or countering forces generated by the stapling device. In one embodiment, the biasing cage includes a plurality of ribbons or strands that extend parallel to the blood flow when expanded in situ. In another embodiment, the biasing cage includes a mesh or braided structure. In another embodiment, the biasing cage includes a plurality of ribbons or strands that extend parallel to the blood flow when expanded in situ and a mesh or braided structure placed over the plurality of ribbons. Further details and description of these embodiments are provided below.

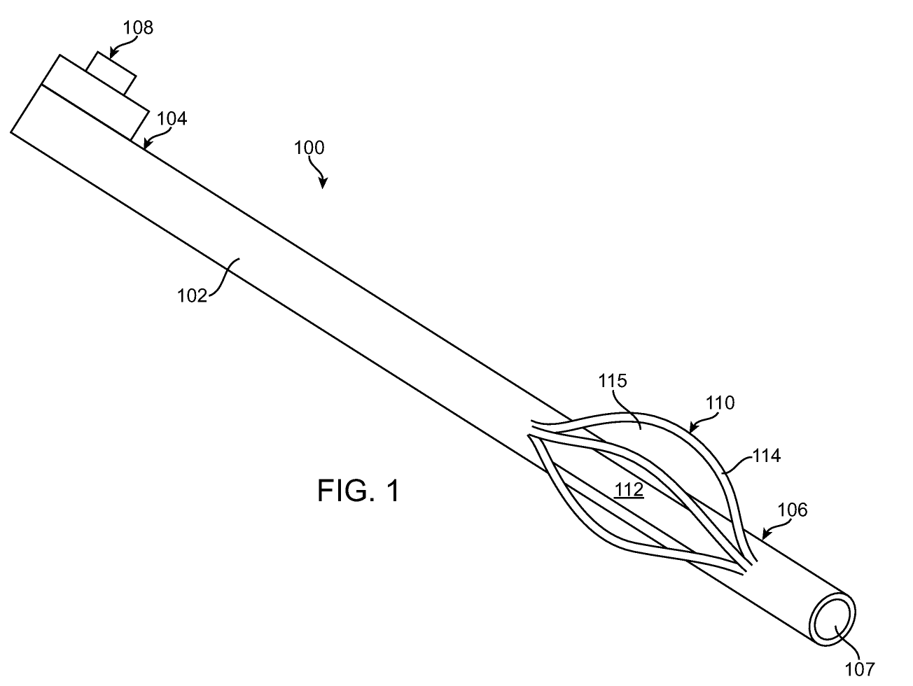

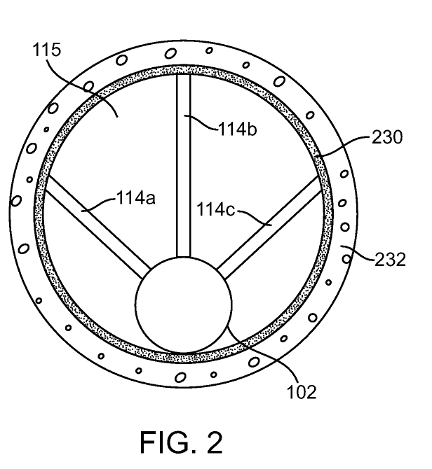

Referring to FIGS. 1-2, an endostapler delivery system 100 includes a catheter shaft 102 having an expandable biasing cage 110 disposed at the distal portion thereof. FIG. 1 is an schematic isometric view of endostapler delivery system 100, while FIG. 2 is an end view of the endostapler delivery system 100 positioned within a vessel for attaching an endoluminal graft to a vessel wall 232. Catheter shaft 102 includes a proximal portion 104 and a distal portion 106, wherein distal portion 106 includes an exit port 107. In addition, as will be explained in more detail below, catheter shaft 102 has at least one lumen extending there through for receiving a stapling device for attaching an endovascular graft 230 to a vessel wall 232 of a body lumen. A side recess or port 112 is provided at the distal portion 106 of catheter shaft 102 for exposing the expandable biasing cage 110. An actuator 108 is provided at the proximal portion 104 of catheter shaft 102 for expanding biasing cage 110 to a dome or semi-circular shape. Biasing cage 110 is expanded to the dome or semi-circular shape in situ in order to ensure that the stapling device abuts the vessel and/or graft. During operation of the stapling device, expanded biasing cage 110 acts as an anchor to offset or counter forces generated by the stapling device.

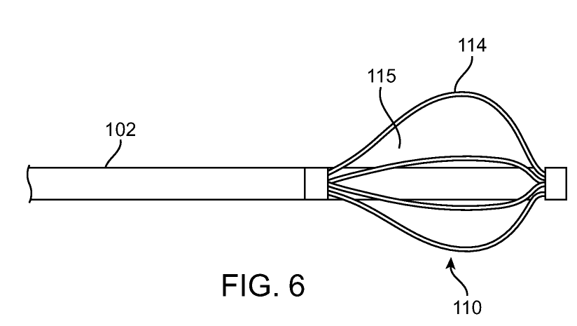

Biasing cage 110 includes a plurality of ribbons or strands 114 that extend generally parallel to the blood flow when expanded. Open spaces 115 disposed between the plurality of ribbons or strands 114 when biasing cage is expanded allow blood or other fluid to flow there through during the stapling procedure such that the blood vessel is not blocked or occluded. In one example shown in FIGS. 1-2 and 6, biasing cage 110 includes three ribbons 114a, 114b, and 114c. However, one of ordinary skill in the art will appreciate that biasing cage 110 may include any number of ribbons or strands. For example, biasing cage 110 may include between two and five ribbons or strands that extend generally parallel to the blood flow when expanded. The plurality of ribbons 114 have sufficient mechanical strength to anchor the catheter shaft 102 to offset or counter forces generated by a stapling device when the stapling device is utilized in securing endovascular graft 230 (only a cross section of which is shown) to a vessel wall 232 of a body lumen. More particularly, biasing cage 110 may be expanded prior to the firing of a staple. Expanding biasing cage 110 forces the stapling device against a receiving area of the vessel wall 232 and/or graft 230 where a staple is to be fired. Preferably, the receiving area of the vessel wall 232 and/or graft 230 is positioned on the opposite side of the vessel to the average centerline of force vectors associated with the expansion of the various components of the biasing cage 110. In addition to placing the stapling device immediately adjacent to the receiving area of the vessel wall 232 and/or graft 230, biasing cage 110 also assists with preventing the stapling device from moving during the firing of the staple.

Embodiments described may be used with any conventional stapling device capable of securing graft 230 to vessel wall 232. Thus, it will be apparent to those of ordinary skill in the art that any features of the stapling device discussed herein are exemplary in nature. For example, the stapling device may be any stapling device known in the art, including but not limited to those shown or described in US Patent Publication 20040176786 assigned to Edrich Vascular, US Patent Publication 20070073389 assigned to Aptus Endosystems, Inc., and US Patent Publication 20070162053 assigned to Anson Medical. In another embodiment (not shown), the stapling device may be an integral part of the biasing endostapler delivery system, i.e., formed as one integral piece within a lumen of the catheter.

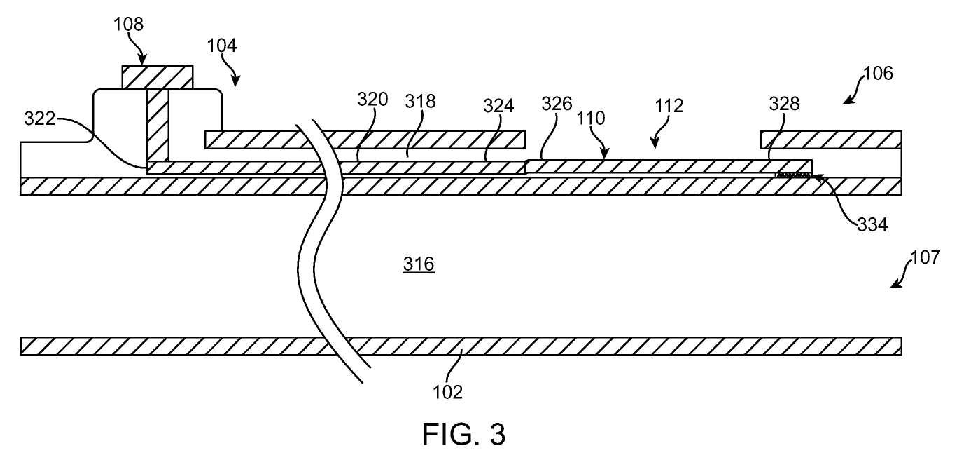

As shown in FIG. 3, catheter shaft 102 is a multi-lumen catheter. FIG. 3 is a sectional side view of the endostapler delivery system 100 illustrated in FIG. 1. Catheter shaft 102 includes a first lumen 316 extending along the entire length thereof for receiving a stapling device. In the present embodiment, first lumen 316 is open-ended and in fluid communication with exit port 107 such that the stapling device may exit out of the exit port 107 at the distal portion 106 of catheter shaft 102. However, as will be explained in greater detail herein, alternatively the first lumen may be closed-ended but in fluid communication with an exit port located in the side of the catheter shaft such that a side-firing stapling device may be used. Catheter shaft 102 also includes a second lumen 318 that extends from the proximal portion 104 to the distal portion 106 of catheter shaft 102 for housing the biasing mechanism, including biasing cage 110. Second lumen 318 is parallel and adjacent to first lumen 316. Second lumen 318 is closed-ended but in fluid communication with side recess or port 112 provided at the distal portion 106 of catheter shaft 102. Side recess or port 112 allows biasing cage 110 to expand and abut the vessel wall 232 and/or graft 230.

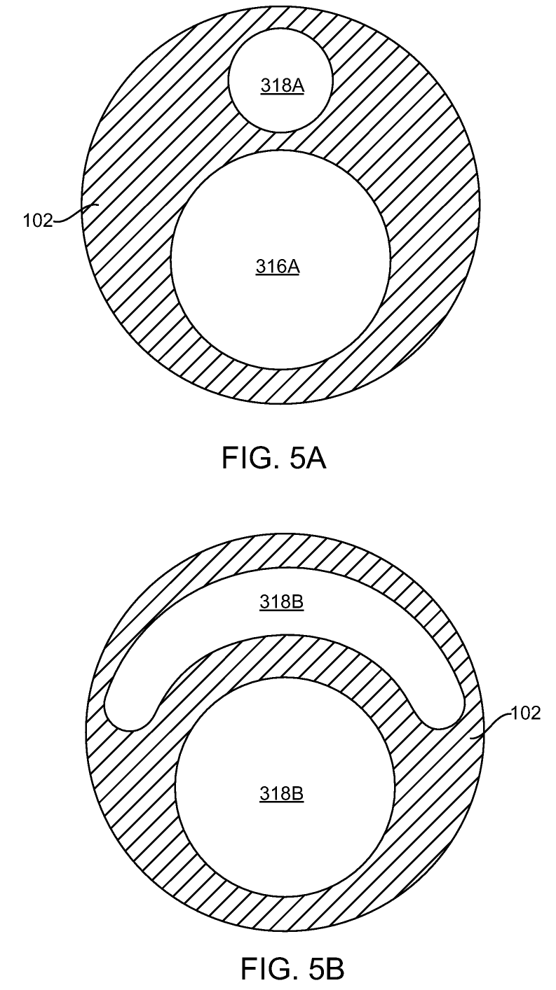

First lumen 316 and second lumen 318 are thus in a side-by-side arrangement through the length of the catheter, and may each have any suitable cross-section. For example, FIG. 5A is a cross-sectional view of endostapler delivery system 100 in accordance with one embodiment in which both first lumen 316A and second lumen 318A have circular or elliptical cross-sections. First lumen 316 of catheter shaft 102 is of a sufficient size to accommodate a stapling device. For example, a conventional stapling typically has a profile or an outer diameter of approximately 4 mm-5 mm (12-15 French units) and thus the diameter of first lumen 316 of catheter shaft 102 should be of a slightly larger size in order to ensure that a conventional stapling device can be advanced through catheter shaft 102. However, second lumen 318 of catheter shaft 102 is relatively smaller than first lumen 316 because second lumen 318 must only be of a sufficient size to accommodate the biasing mechanism, including unexpanded biasing cage 110. It is desirable to keep second lumen 318 as small as possible in order to minimize the outer diameter of catheter shaft 102, thus minimizing the size of endostapler delivery system 100 such that endostapler delivery system 100 may fit within relatively small vessels. The outer diameter of the catheter shaft may be approximately 3 mm-8 mm.

Other embodiments of catheter shaft 102 may have first lumen 316 and second lumen 318 in other dual lumen arrangements, such as a kidney or arc-shaped second lumen above a circular first lumen as shown in FIG. 5B. FIG. 5B is a cross-sectional view of the endostapler delivery system illustrated in FIG. 1 in accordance with another embodiment. Another alternative dual lumen arrangement is a crescent-shaped second lumen above a circular first lumen (not illustrated). As described above, the only limitation on the cross-sectional shapes of first lumen 316 and second lumen 318 is that first lumen 316 must be a sufficient size to accommodate a stapling device and second lumen 318 must be of a sufficient size to accommodate the biasing mechanism.

While not shown in any of the figures, the use of an outer cover, catheter outer sheath may be employed to provide a continuous smooth and slick (e.g., lubricious hydrophilic coating coated) surface to facilitate easy introduction of the catheter into the patient. Once the end of the catheter has been positioned near the delivery location, the outer cover is drawn back, either by the closing of a gap at the handle, or by splitting the outer sheath and having at least a proximal portion of it constructed as a peel away type sheath.

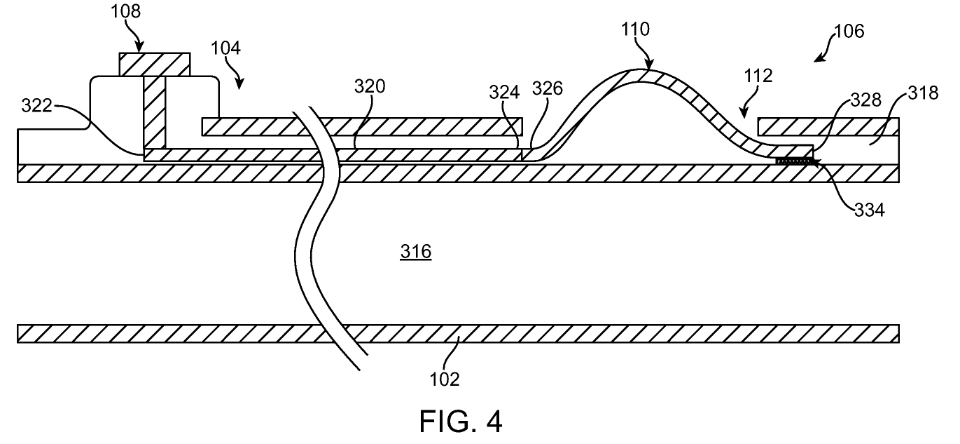

Referring now to FIGS. 3-4, biasing cage 110 is movable from an unexpanded position (shown in FIG. 3) to an expanded position (shown in FIG. 4). In the unexpanded position, biasing cage 110 is relatively straight in order to minimize the delivery profile as endostapler delivery system 100 is advanced to a position within graft 230. Further, in the unexpanded position, biasing cage 110 is completely housed within second lumen 318. Biasing cage 110 is then expanded via actuator 108 to the expanded position shown in FIG. 4, as well as FIGS. 1, 2 and 6. In the expanded position, biasing cage 110 assumes a dome or semi-circular shape extending outside of catheter shaft 102 via side recess or port 112 such that biasing cage 110 abuts the vessel wall 232 and/or graft 230. Thus, the height of the expanded biasing cage 110 must be sufficient to enable the biasing cage 110 to abut the vessel wall 232 and/or graft 230. For example, a target vessel lumen may be approximately 36 mm in diameter. Accordingly, if the outer diameter of catheter shaft 102 is approximately 3 mm-8 mm, the deployment height of the expanded biasing cage (that is, the height of the dome or semi-circular shape extending outside of catheter shaft 102) should be approximately 12 mm-30 mm or of a slightly larger size in order to ensure that the expanded biasing cage 110 abuts the vessel wall 232 and/or graft 230.

As shown in FIGS. 3 and 4, to expand biasing cage 110, actuator 108 may be a turning or push-pull actuator (i.e., a knob or handle) that is attached or connected to a rod 320 which extends through second lumen 318. Rod 320 has a proximal end 322 and a distal end 324, the proximal end 322 being connected to actuator 108 and the distal end 324 being connected to a proximal end 326 of biasing cage 110. A distal end 328 of biasing cage 110 is fixed via a connection 334 to catheter shaft 102. When actuator 108 is operated (i.e., manually turned or pushed), rod 320 is advanced through second lumen 318 of catheter shaft 102. Since distal end 328 of biasing cage 110 is fixed, biasing cage 110 expands or deploys to the expanded dome or semi-circular shape when the material of biasing cage 110 radially expands via side recess or port 112. In another embodiment, biasing cage 110 may extend through the entire second lumen 318 of catheter 102 such that the proximal end 326 of the biasing cage 110 is connected to the actuator 108, thus eliminating the need for rod 320.

Distal end 328 of biasing cage 110 may be attached to catheter shaft 102 in any suitable manner known in the art. For example, connection 334 may be formed by welding, such as by resistance welding, friction welding, laser welding or another form of welding such that no additional materials are used to connect biasing cage 110 to catheter shaft 102. Alternatively, biasing cage 110 and catheter shaft 102 can be connected by soldering, by the use of an adhesive, by the addition of a connecting element there between, or by another mechanical method.

In order to expand or deploy biasing cage 110, endostapler delivery system 100 must be tracked to and properly positioned at implanted endoluminal graft 230. In general, a guidewire (not shown) is introduced into the target vessel. Endostapler delivery system 100 is then tracked over the guidewire such that the exit port 107 is adjacent to the implanted endoluminal graft 230. Once endostapler delivery system 100 is in place as desired, the guidewire may be removed and a conventional stapling device is inserted through first lumen 316 and exit port 107 of catheter shaft 102 and tracked to a position in which the stapling device is adjacent a receiving area of the vessel wall 232 and/or graft 230 where a staple is to be fired. With the guidewire removed, endostapler delivery catheter acts as a guide catheter for tracking the conventional stapling device to the site of the implanted endoluminal graft 230. Alternatively, if the stapling device is an over the wire type device, the guidewire may be left in place within endostapler delivery system 100 and the stapling device may inserted through catheter shaft 102 and tracked over the guidewire. Alternately, the endostapler delivery catheter can be constructed with an additional lumen for a guide wire.

Once the stapling device is in place (that is, adjacent a receiving area of the vessel wall 232 and/or graft 230 where a staple is to be fired), biasing cage 110 may be expanded or deployed in order to maintain the desired position. Expansion of biasing cage 110 pushes the stapling portion of the stapling device against the vessel wall 232 and/or graft 230 where a staple is to be fired. When the staple is fired from the stapling device, biasing cage 110 remains expanded so that it prevents the stapling device from moving during the firing of the staple. Following each staple deployment, biasing cage 110 may be partially or fully collapsed to the unexpanded position. The stapling device is rotated to a second position in preparation for the firing of a second or subsequent staple, and the process is repeated to deploy the next staple. Prior to firing the second or subsequent staple, biasing cage 110 is expanded to place the stapling portion of the stapling device in position in preparation for firing. Once all the staples have been delivered and graft 230 is secured as desired, biasing cage 110 is fully collapsed to the unexpanded position. The stapling device and endostapler delivery system 100 are retracted and removed from the patient. Although methods of using specific embodiments are described herein for securing an endoluminal graft to a vessel wall, it will be apparent to those of ordinary skill in the art that such embodiments may also be utilized for securing extraluminal or transluminal grafts to a vessel wall.

Ribbons 114 of biasing cage 110 are preferably constructed of biocompatible materials having good mechanical strength. For example, non-exhaustive examples of metallic materials for ribbons 114 are stainless steel, cobalt based alloys (605L, MP35N), titanium, tantalum, tungsten based alloys, superelastic nickel-titanium alloy, other biocompatible metals, thermoplastic polymers, or combinations of any of these.

The catheter shaft may be an extruded multi-lumen shaft formed of any suitable flexible polymeric material. Non-exhaustive examples of material for the catheter shaft are polyethylene terephalate (PET), nylon, polyethylene, PEBAX, or combinations of any of these, either blended or co-extruded. Optionally, a portion of the catheter shaft may be formed as a composite having a reinforcement material incorporated within a polymeric body in order to enhance strength, flexibility, and/or toughness. Suitable reinforcement layers include braiding, wire mesh layers, embedded axial wires, embedded helical or circumferential wires, and the like. In an embodiment, the proximal portion of the catheter shaft may in some instances be formed from a reinforced polymeric tube, for example, as shown and described in U.S. Pat. No. 5,827,242 to Follmer et al. which is incorporated by reference herein in its entirety. The catheter shaft may have any suitable working length, for example, 550 mm-650 mm, in order to extend to a target location where a staple is to be fired.

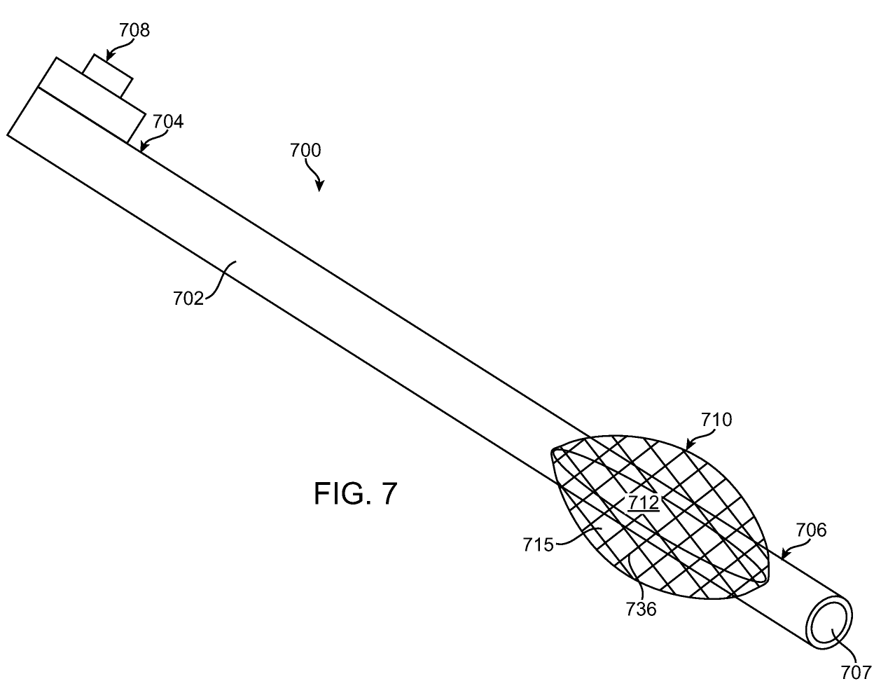

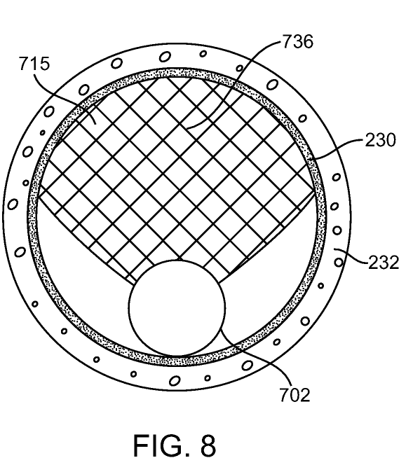

As previously discussed, embodiments described relate to a biasing mechanism to ensure that the stapling portion of the stapling device is secure against a vessel wall and/or graft. Another embodiment of a biasing device which may be utilized for this purpose is shown in FIGS. 7-10. Referring to FIGS. 7 and 8, an endostapler delivery system 700 includes a catheter shaft 702 having an expandable biasing cage 710 at the distal portion thereof. FIG. 7 is an isometric view of endostapler delivery system 700, and FIG. 8 is a front view of the endostapler delivery system 700 utilized within a vessel for attaching an endoluminal graft to a vessel wall. Catheter shaft 702 includes a proximal portion 704 and a distal portion 706, wherein distal portion 706 includes an exit port 707. A side recess or port 712 is provided at the distal portion 706 of catheter shaft 702 for exposing an expandable biasing cage 710. An actuator 708 is provided at the proximal portion 704 of catheter shaft 702 for expanding biasing cage 710 to a dome or semi-circular shape. Biasing cage 710 is expanded to the dome or semi-circular shape in situ in order to ensure that a stapling device inserted through catheter shaft 702 abuts a vessel and/or graft. The stapling device may be any conventional stapling device capable of securing graft 230 to vessel wall 232.

Biasing cage 710 includes a braided structure or mesh 736. Open spaces 715 disposed within mesh 736 when biasing cage 710 is expanded allow blood or other fluid to flow through the vessel during the stapling procedure. The braided structure or mesh 736 has sufficient mechanical strength to offset or counter forces generated by a stapling device when the stapling device is utilized in securing endovascular graft 230 to a vessel wall 232 of a body lumen. More particularly, biasing cage 710 may be expanded prior to the firing of a staple. Expanding biasing cage 710 forces the stapling device against a receiving area of a vessel wall 232 and/or graft 230 where a staple is to be fired. Preferably, the receiving area of the vessel wall 232 and/or graft 230 is positioned on the opposite side of the vessel than biasing cage 710. In addition to placing the stapling device immediately adjacent to the receiving area of the vessel wall 232 and/or graft 230, biasing cage 710 also assists with preventing the stapling device from moving during the firing of the staple.

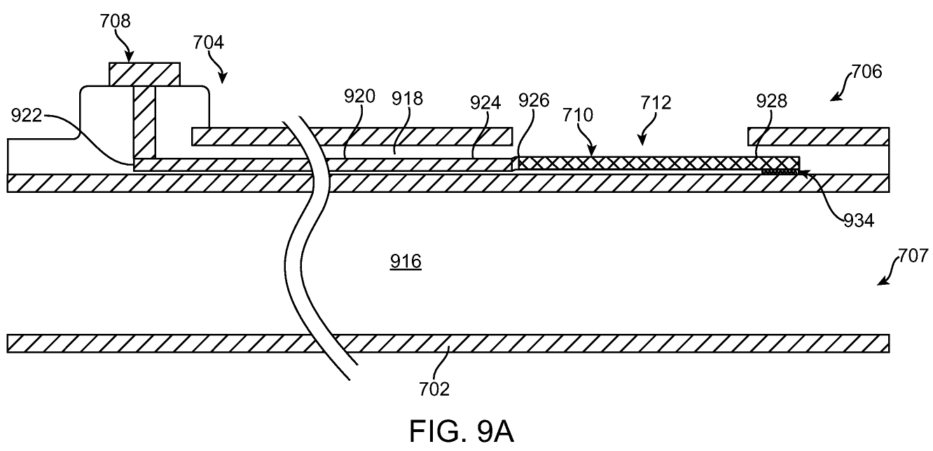

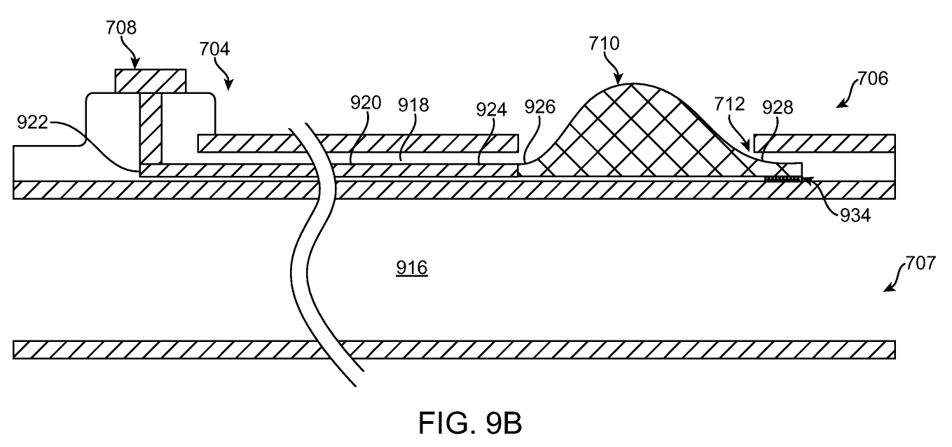

As shown in FIG. 9A, catheter shaft 702 is a multi-lumen catheter. FIG. 9A is a sectional side view of the endostapler delivery system illustrated in FIG. 7. Catheter shaft 702 includes a first lumen 916 extending along the entire length thereof for receiving a stapling device. First lumen 916 is open-ended such that it is in fluid communication with exit port 707 such that stapling device may exit out of the exit port 707 of catheter shaft 702. However, as will be explained in greater detail herein, alternatively the first lumen may be closed-ended but in fluid communication with an exit port located in the side of the catheter shaft such that a side-firing stapling device may be used. Catheter shaft 702 also includes a second lumen 918 that extends from the proximal portion 704 to the distal portion 706 of catheter shaft 702 for housing the biasing mechanism, including biasing cage 710. Second lumen 918 is parallel and adjacent to first lumen 916. Second lumen 918 is closed-ended but in fluid communication with side recess or port 712 provided at the distal portion 706 of catheter shaft 702. Side recess or port 712 allows biasing cage 710 to expand and abut the vessel wall 232 and/or graft 230. As described above with respect to previous embodiments, first lumen 916 of catheter shaft 702 is of a sufficient size to accommodate a stapling device and second lumen 918 is of a sufficient size to accommodate the biasing mechanism, including biasing cage 710. First lumen 916 and second lumen 918 may each have any suitable cross-section such as those described with respect to previous embodiments.

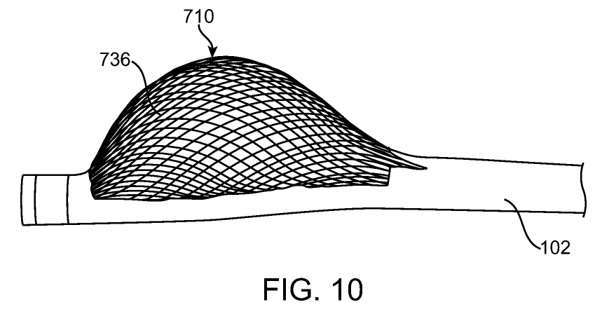

Referring now to FIGS. 9A-9B, biasing cage 710 is movable from an unexpanded position (shown in FIG. 9A) to an expanded position (shown in FIG. 9B). In the unexpanded position, biasing cage 710 is relatively straight in order to minimize the delivery profile as endostapler delivery system 700 is advanced to graft 230. Further, in the unexpanded position, biasing cage 710 is completely housed with second lumen 918. Biasing cage 710 is then expanded via actuator 708 to the expanded position shown in FIGS. 9B and 10. In the expanded position, biasing cage 710 assumes a dome or semi-circular shape extending outside of catheter shaft 702 via side recess or port 712 such that biasing cage 710 abuts the vessel wall 232 and/or graft 230. Thus, the height of the expanded biasing cage 710 must be sufficient to enable the biasing cage 710 to abut the vessel wall 232 and/or graft 230. In order to expand biasing cage 710, actuator 708 may be a rotational (to be turned) or push-pull actuator (i.e., a knob or handle) that is attached or connected to a rod 920 which extends through second lumen 918. Rod 920 includes a proximal end 922 and a distal end 924, the proximal end 922 being connected to actuator 708 and the distal end 924 being connected to a proximal end 926 of biasing cage 710. A distal end 928 of biasing cage 710 is fixed via a connection 934 to catheter shaft 702. Distal end 928 of biasing cage 710 may be attached to catheter shaft 702 in any suitable manner known in the art as described above with respect to previous embodiments. When actuator 708 is operated (i.e., manually, turned, rotated, or pushed), rod 920 is advanced through second lumen 918 of catheter shaft 702. Since distal end 928 of biasing cage 710 is fixed, biasing cage 710 expands or deploys to the expanded dome or semi-circular shape when the material of biasing cage 710 radially expands via side recess or port 712. In another embodiment, biasing cage 710 may extend through the entire second lumen 918 of catheter 702 such that the proximal end 926 of the biasing cage 710 is connected to the actuator 708, thus eliminating the need for rod 920.

Mesh 736 (shown in FIG. 10) of biasing cage 710 is preferably constructed of implantable polymeric or metallic materials having good mechanical strength. Non-exhaustive examples of polymeric materials for mesh 736 are polyurethane, polyethylene terephalate (PET), nylon, polyethylene, PEBAX, or combinations of any of these, either blended or co-extruded. Non-exhaustive examples of metallic materials for mesh 736 are stainless steel, cobalt based alloys (605L, MP35N), titanium, tantalum, superelastic nickel-titanium alloy, or combinations of any of these.

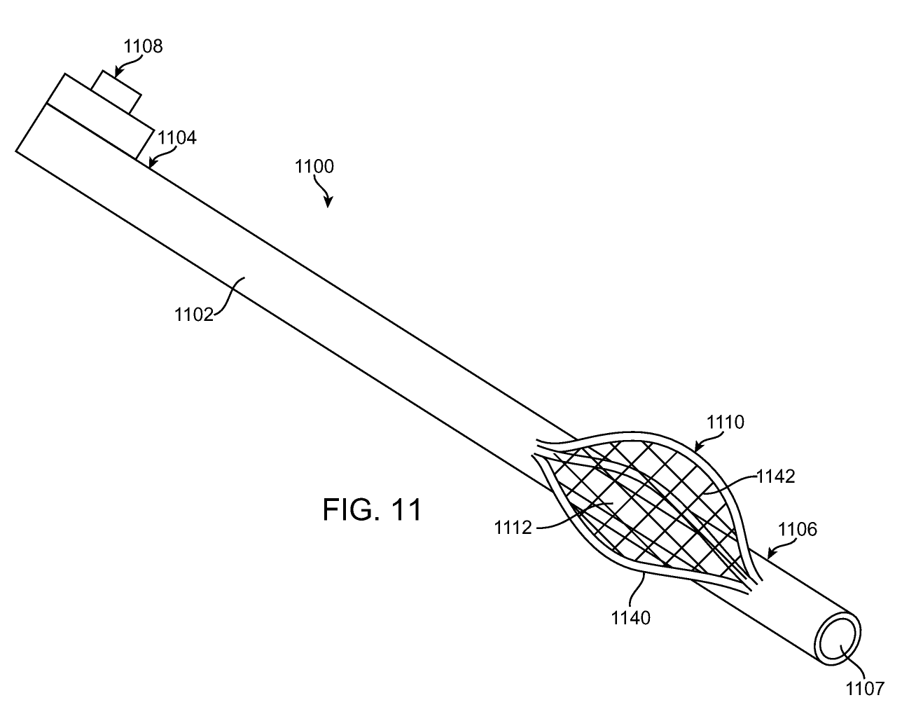

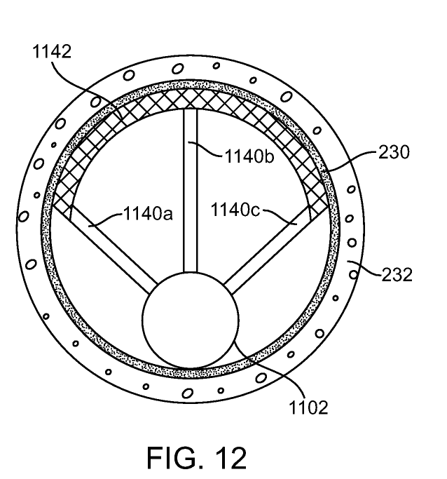

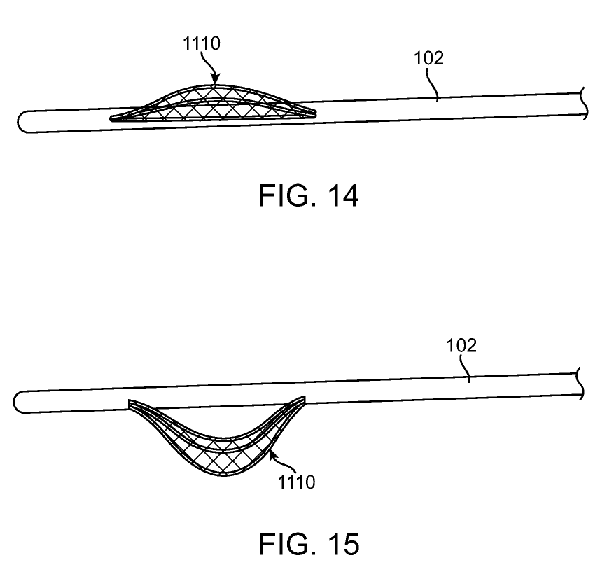

As previously discussed, the embodiments described relate to a biasing mechanism to ensure that the stapling portion of the stapling device is secure (anchored) against a vessel wall and/or graft. Another embodiment of a biasing device which may be utilized for this purpose is shown in FIGS. 11-15. Referring to FIGS. 11 and 12, an endostapler delivery system 1100 includes a catheter shaft 1102 having an expandable biasing cage 1110 at a distal portion thereof. FIG. 11 is a schematic isometric view of endostapler delivery system 1100, and FIG. 12 is a front view of the endostapler delivery system 1100 utilized within a vessel for attaching an endoluminal graft 230 to a vessel wall 232. Catheter shaft 1102 includes a proximal portion 1104 and a distal portion 1106, wherein distal portion 1106 includes an exit port 1107. A side recess or port 1112 is provided at the distal portion 1106 of catheter shaft 1102 for exposing an expandable biasing cage 1110. An actuator 1108 is provided at the proximal portion 1104 of catheter shaft 1102 for expanding biasing cage 1110 to a dome or semi-circular shape. Biasing cage 1110 is expanded to the dome or semi-circular shape in situ in order to ensure that a stapling device inserted through catheter shaft 1102 abuts a vessel wall 232 and/or a graft 230. The stapling device may be any conventional stapling device capable of securing graft 230 to vessel wall 232.

Biasing cage 1110 includes a plurality of ribbons or strands 1140 that extend generally parallel to the blood flow when expanded, and includes a braided structure or mesh 1142 placed over the plurality of ribbons 1140. Biasing cage 1110 does not block or occlude a vessel and thus allows blood or other fluid to flow there through during the stapling procedure. In one example shown in FIGS. 11-15, biasing cage 1110 includes three ribbons 1140a, 1140b, and 1140c. However, one of ordinary skill in the art will appreciate that biasing cage 1110 may include any number of ribbons or strands. For example, biasing cage 1110 may include between two and five ribbons or strands that extend generally parallel to the blood flow when expanded. In this embodiment, the plurality of ribbons 1140 have sufficient mechanical strength to offset or counter forces generated by a stapling device when the stapling device is utilized in securing endovascular graft 230 to a vessel wall 232 of a body lumen while mesh 1142 provides atraumatic gentle contact with a vessel wall. More particularly, biasing cage 1110 may be expanded prior to the firing of a staple. Expanding biasing cage 1110 forces the stapling device against a receiving area of a vessel wall 232 and/or graft 230 where a staple is to be fired. Preferably, the receiving area of the vessel wall 232 and/or graft 230 is positioned on the opposite side of the vessel than biasing cage 1110. In addition to placing the stapling device immediately adjacent to the receiving area of the vessel wall 232 and/or graft 230, biasing cage 1110 also assists with preventing the stapling device from moving during the firing of the staple.

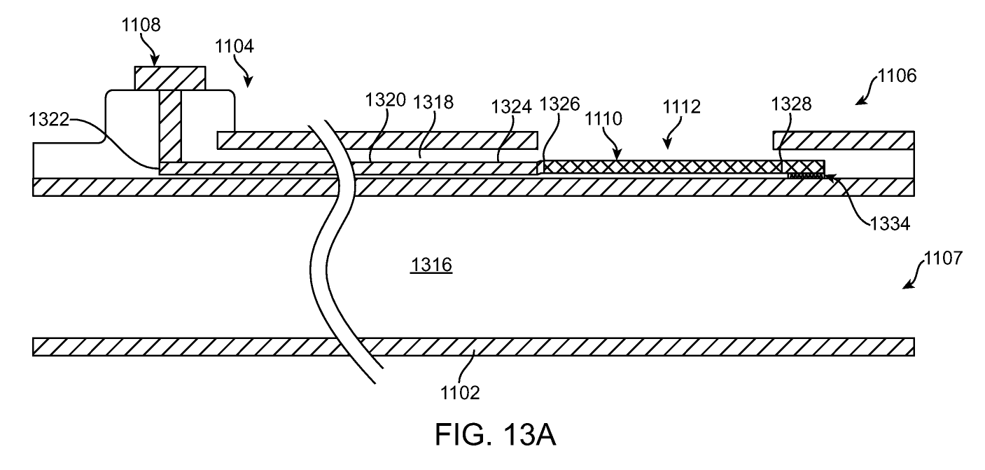

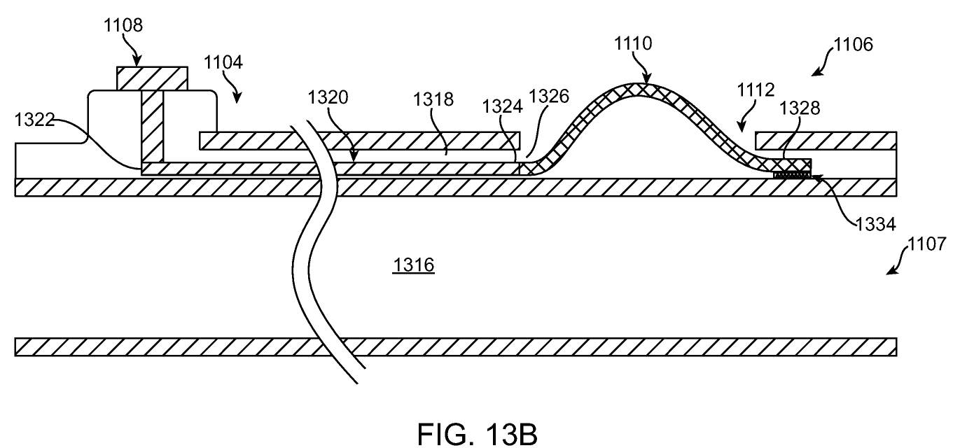

As shown in FIG. 13A, catheter shaft 1102 is a multi-lumen catheter. FIG. 13A is a sectional side view of the endostapler delivery system illustrated in FIG. 11. Catheter shaft 1102 includes a first lumen 1316 extending along the entire length thereof for receiving a stapling device. First lumen 1316 is open-ended and in fluid communication with exit port 1107 such that the stapling device may exit out of the exit port 1107 of catheter shaft 1102. However, as will be explained in greater detail herein, alternatively the first lumen may be closed-ended but in fluid communication with an exit port located in the side of the catheter shaft such that a side-firing stapling device may be used. Catheter shaft 1102 also includes a second lumen 1318 that extends from the proximal portion 1104 to the distal portion 1106 of catheter shaft 1102 for housing the biasing mechanism, including biasing cage 1110. Second lumen 1318 is parallel and adjacent to first lumen 1316. Second lumen 1318 is closed-ended but in fluid communication with side recess or port 1112 provided at the distal portion 1106 of catheter shaft 1102. Side recess or port 1112 allows biasing cage 1110 to expand and abut the vessel wall 232 and/or graft 230. As described above with respect to previous embodiments, first lumen 1316 of catheter shaft 1102 is of a sufficient size to accommodate a stapling device and second lumen 1318 is of a sufficient size to accommodate the biasing mechanism, including biasing cage 1110. First lumen 1316 and second lumen 1318 may each have any suitable cross-section such as those described with respect to previous embodiments.

Referring now to FIGS. 13A-13B, biasing cage 1110 is movable from an unexpanded position (shown in FIG. 13A) to an expanded position (shown in FIG. 13B). In the unexpanded position, biasing cage 1110 is relatively straight in order to minimize the delivery profile as endostapler delivery system 1100 is advanced to graft 230. Further, in the unexpanded position, biasing cage 1110 is completely housed with second lumen 1318. Biasing cage 1110 is then expanded via actuator 1108 to the expanded position shown in FIGS. 13B and 14-15. In the expanded position, biasing cage 1110 assumes a dome or semi-circular shape extending outside of catheter shaft 1102 via side recess or port 1112 such that biasing cage 1110 abuts the vessel wall 232 and/or graft 230. Thus, the height of the expanded biasing cage 1110 must be sufficient to enable the biasing cage 1110 to abut the vessel wall 232 and/or graft 230. To expand biasing cage 1110, actuator 1108 may be a rotational (to be turned) or push-pull actuator (i.e., a knob or handle) that is attached or connected to a rod 1320 which extends through second lumen 1318. Rod 1320 has a proximal end 1322 and a distal end 1324, the proximal end 1322 being connected to actuator 1108 and the distal end 1324 being connected to a proximal end 1326 of biasing cage 1110. A distal end 1328 of biasing cage 1110 is fixed via a connection 1334 to catheter shaft 1102. Distal end 1328 of biasing cage 1110 may be attached to catheter shaft 1102 in any suitable manner known in the art as described above with respect to previous embodiments. When actuator 1108 is operated (i.e., manually, turned, rotated, or pushed), rod 1320 is advanced through second lumen 1318 of catheter shaft 1102. Since second distal end 1328 of biasing cage 1110 is fixed, biasing cage 1110 expands or deploys to the expanded dome or semi-circular shape when the material of biasing cage 1110 radially expands via side recess or port 1112. In another embodiment, biasing cage 1110 may extend through the entire second lumen 1318 of catheter 1102 such that the proximal end 1326 of the biasing cage 1110 is connected to the actuator 1108, thus eliminating the need for rod 1320.

Mesh 1142 is positioned or superimposed over ribbons 1140 to form biasing cage 1110. Mesh 1142 of biasing cage 1110 provides atraumatic gentle contact with the vessel wall and thus is preferably constructed of a flexible implantable polymeric material. Non-exhaustive examples of polymeric materials for mesh 1142 are polyurethane, polyethylene terephalate (PET), nylon, polyethylene, PEBAX, or combinations of any of these, either blended or co-extruded. Ribbons 1140 have sufficient mechanical strength to offset or counter forces generated by a stapling device and thus are preferably constructed from an implantable metallic material having good mechanical strength. Non-exhaustive examples of metallic materials for ribbons 1140 are stainless steel, cobalt based alloys (605L, MP35N), titanium, tantalum, superelastic nickel-titanium alloy, or combinations of any of these.

Biasing cage 1110 having a combination of a plurality of ribbons or strands 1140 and a braided structure or mesh 1142 would have an advantage of a smaller delivery profile. Ribbons 1140 act as the structural element in that they provide the majority of the structural support needed to assure catheter contact with the vessel wall. Ribbons 1140 can be constructed with a narrower cross sectional configuration to minimize catheter crossing profile, as the adjacent mesh structure 1142 will distribute the force exerted over a larger area than just the surface of the ribbons and as such will provide a combined element that provides atraumatic contact with the vessel wall. The general understood means of forming such shape memory ribbons would be used to shape the ribbon to pre-set shape expanded predetermined diameter. In operation, a push pull and/or screw actuation mechanism would then be used for deployment.

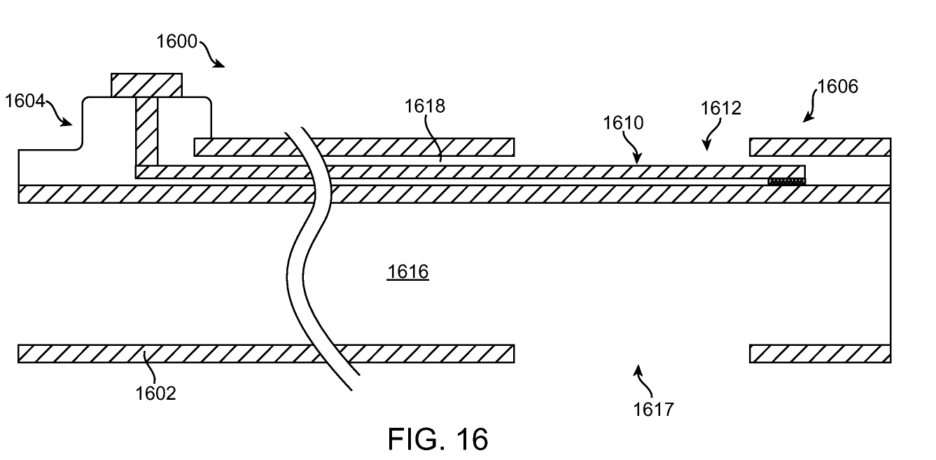

As previously described, the first lumen of the catheter shaft that receives the stapling device may be open-ended and in fluid communication with an exit port such that the stapling device may exit out of the distal open-ended exit port. Alternatively, the first lumen of the catheter shaft may be closed-ended but in fluid communication with an exit port located in the side of the catheter shaft such that a side-firing stapling device may be utilized. For example, as shown in FIG. 16, catheter shaft 1602 is a multi-lumen catheter including a first lumen 1616 extending along the entire length thereof for receiving a stapling device. First lumen 316 is closed-ended but in fluid communication with side exit port 1617 of the catheter such that a side-firing stapling device may exit out of the side exit port 1617 at the distal portion 1606 of catheter shaft 102. Similar to previously described embodiments, catheter shaft 1602 also includes a second lumen 1618 that extends from the proximal portion 1604 to the distal portion 1606 of catheter shaft 1602 for housing the biasing mechanism, including biasing cage 1610. Second lumen 1618 is parallel and adjacent to first lumen 1616. Second lumen 1618 is closed-ended but in fluid communication with side recess or port 1612 provided at the distal portion 1606 of catheter shaft 1602 to allow biasing cage 1610 to expand and abut the vessel wall and/or graft. Distal side exit port 1617 is located directly across from (on the opposite side of the catheter shaft) side recess or port 1612 so that a staple is fired directly opposite from the approximate centerline of an expanded portion of the biasing mechanism (biasing cage 1610).

While various embodiments according to the present invention have been described above, it should be understood that they have been presented by way of illustration and example only, and not limitation. It will be apparent to persons skilled in the relevant art that various changes in form and detail can be made therein without departing from the spirit and scope of that described. It will also be understood that each feature of each embodiment discussed herein, and of each reference cited herein, can be used in combination with the features of any other embodiment. All patents and publications discussed herein are incorporated by reference herein in their entirety.

What is claimed is:

1. An endostapler delivery system for delivering a stapling device through a body lumen, comprising:
a catheter shaft including a proximal portion and a distal portion, the catheter shaft defining a first lumen having an exit port disposed at the distal portion of the catheter shaft and a second lumen having a side exit port disposed at the distal portion of the catheter shaft, wherein the first and second lumens extend side-by-side from the proximal portion to the distal portion of the catheter shaft, and wherein the first lumen of the catheter shaft is of a sufficient size such that the stapling device may be advanced there through;
an expandable biasing cage disposed within the second lumen of the catheter shaft; and
an actuator disposed at the proximal portion of the catheter shaft, wherein the actuator is adapted to expand the biasing cage to a dome shape extending outside of the catheter shaft via the side exit port such that the biasing cage abuts a vessel wall of the body lumen and/or a graft implanted within the body lumen,
wherein the biasing cage when expanded does not block or occlude the body lumen such that blood may flow there through.

2. The endostapler delivery system of claim 1, wherein the biasing cage has a straightened unexpanded configuration that lies completely within the second lumen of the catheter shaft.

3. The endostapler delivery system of claim 1, wherein the actuator is selected from the group consisting of a push-pull actuator and a turning actuator.

4. The endostapler delivery system of claim 3, wherein a distal end of the biasing cage is fixed within the second lumen of the catheter shaft such that when the actuator is operated, the biasing cage expands radially via the side exit port.

5. The endostapler delivery system of claim 1, wherein the exit port of the first lumen is located is a side port located in the side of the catheter shaft.

6. The endostapler delivery system of claim 5, wherein the exit port of the first lumen is located directly across from the side exit port of the second lumen.

7. The endostapler delivery system of claim 1, wherein first lumen is open-ended at a distal end and the exit port of the first lumen is located at the open-ended distal end of the catheter shaft.

8. The endostapler delivery system of claim 1, wherein the biasing cage is formed from a plurality of ribbons that extend parallel to the blood flow such that the biasing cage when expanded does not block or occlude the body lumen such that blood may flow there through.

9. The endostapler delivery system of claim 8, wherein the plurality of ribbons includes three ribbons.

10. The endostapler delivery system of claim 8, wherein the plurality of ribbons are constructed from a material selected from the group consisting of stainless steel, a cobalt alloy, titanium, titanium alloys, tantalum, tantalum alloys, a nickel-titanium alloy, and tungsten alloys.

11. The endostapler delivery system of claim 1, wherein the biasing cage is formed from a mesh structure such that the biasing cage when expanded does not block or occlude the body lumen such the blood may flow there through.

12. The endostapler delivery system of claim 11, wherein the mesh structure is constructed from a material selected from the group consisting of stainless steel, a cobalt alloy, titanium, titanium alloys, tantalum, tantalum alloys, a nickel-titanium alloy, and tungsten alloys.

13. The endostapler delivery system of claim 1, wherein the biasing cage is formed from a plurality of ribbons that extend generally parallel to the blood flow and a mesh structure disposed over the plurality of ribbons such that the biasing cage when expanded does not block or occlude the body lumen such that blood may flow there through.

14. The endostapler delivery system of claim 13, wherein the plurality of ribbons includes three ribbons.

15. The endostapler delivery system of claim 14, wherein the plurality of ribbons are constructed from a material selected from the group consist stainless steel, a cobalt alloy, titanium, titanium alloys, tantalum, tantalum alloys, a nickel-titanium alloy, and tungsten alloys.

16. The endostapler delivery system of claim 13, wherein the mesh is constructed from a polymeric material.

17. A method of delivering a stapling device through a body lumen, the method comprising the steps:
tracking an endostapler delivery system to a target location within the body lumen, wherein the endostapler delivery system includes
a catheter shaft having a proximal portion and a distal portion, the catheter shaft defining a first lumen having an exit port disposed at the distal portion of the catheter shaft and a second lumen having a side exit port disposed at the distal portion of the catheter shaft, wherein the first and second lumens extend side-by-side from the proximal portion to the distal portion of the catheter shaft,
an expandable biasing cage disposed within the second lumen of the catheter shaft, and
an actuator provided at the proximal portion of the catheter shaft;
tracking the stapling device through the first lumen of the endostapler delivery system such that the stapling device is adjacent to the target location within the body;
operating the actuator in order to expand the biasing cage to a dome shape extending outside of the catheter shaft via the side exit port such that the biasing cage abuts a vessel wall of the body lumen and/or a graft implanted within the body lumen, wherein the biasing cage when expanded does not block or occlude the body lumen such that blood may flow there through; and
firing a staple from the stapling device, wherein the expanded biasing cage operates to prevent or reduce movement of the stapling device during firing of the staple.

18. The method of claim 17, wherein the target location within the body lumen is an endovascular graft.

19. The method of claim 17, wherein the exit port of the first lumen is located is a side port located in the side of the catheter shaft.

20. The method of claim 19, wherein the exit port of the first lumen is located directly across from the side exit port of the second lumen.

21. The method of claim 17, wherein first lumen is open-ended at a distal end and the exit port of the first lumen is located at the open-ended distal end of the catheter shaft.

22. The method of claim 17, wherein the biasing cage is formed from a plurality of ribbons that extend parallel to the blood flow such that the biasing cage when expanded does not block or occlude the body lumen such that blood may flow there through.

23. The method of claim 17, wherein the biasing cage is formed from a mesh structure such that the biasing cage when expanded does not block or occlude the body lumen such the blood may flow there through.

24. The method of claim 17, wherein the biasing cage is formed from a plurality of ribbons that extend generally parallel to the blood flow and a mesh structure disposed over the plurality of ribbons such that the biasing cage when expanded does not block or occlude the body lumen such that blood may flow there through.

25. The method of claim 24, wherein the mesh is constructed from a polymeric material.
